(19) United States
(12) Patent Application Publication
(10) Pub. No.: US 2005/0027231 A1
(43) Pub. Date: Feb. 3, 2005

(54) Mobile heart-lung machine

(75) Inventor: Karsten Kirchhof, Burghaun-Rothenkirchen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Lifebridge medizintechnik GmbH, Munchen (DE)

(21) Appl. No.: 10/923,730

(22) Filed: Aug. 24, 2004

Related U.S. Application Data
(63) Continuation of application No. 09/890,281, filed on Oct. 5, 2001, now abandoned.

(30) Foreign Application Priority Data
Feb. 12, 1999 (DE) 199 05 937.3

(51) Int. Cl. A61M 37/00

(52) U.S. Cl. 604/4.01

(57) ABSTRACT

The invention relates to a mobile heart-lung machine for maintaining circulation by taking over or supporting the cardiopulmonary function, with at least one venous tube (5), a blood pump (8), an oxygenator (9), an arterial filter (15), an arterial tube (4), and also a tubular blood-circulating system (7), an automatic control system (24) and a self-sufficient voltage supply. In order that the heart-lung machine can be handled in a simple way, the elements (4, 5, 6, 7, 8, 9, 15, 18, 21) which circulate the blood, receive the biochemical and physiological signals and carry out the control commands on the one hand and the drive and automatic control elements (23, 24, 27, 28, 29, 30, 31, 32) on the other hand are arranged in two separate modules (2, 3) and can be joined together to form one functional unit.

The invention relates to a mobile heart-lung machine for maintaining circulation according to the preamble of claim 1.

For maintaining a person's circulation, anoxemic venous blood is removed from the person by means of a heart-lung machine with a cannula and fed by a blood pump to an oxygenator to avoid or eliminate a circulatory arrest or lack of perfusion during an operation as the result of an accident or some other organ failure. In the oxygenator, which performs the function of an artificial lung, the blood is enriched with oxygen and CO₂ is removed. Subsequently, the oxygen-rich, arterial blood is fed back to the patient's circulation through a cannula after cleaning in an arterial filter. Heart-lung machines of this type are used for stationary applications in hospitals. Such a stationary heart-lung machine is described for example in WO 99/59654 (CARDIOVENTION). It comprises an integrated oxygenator and pump unit, arranged in the sterile area around the patient, and a control console outside the sterile area and connected to said unit via a connecting cable. The control console has control devices and drives for the pump and oxygenator.

DE 43 43 334 A1 discloses a heart-lung machine for mobile use. It has a supporting structure, provided with carrying handles at the front and back, with standing feet protruding downward. For fixing the component parts of the heart-lung machine, corresponding fastening means are provided. In the case of this machine, the functionally important elements are arranged such that they are freely accessible and are consequently not protected against damage, which may impair serviceability during use. In addition, the machine must always be carried by two people.

A mobile heart-lung machine is also disclosed by DE 197 02 098 A1. This comprises a loop for feeding blood to an artery, a second loop for removing blood from a vein, a venous reservoir, an oxygenator, a blood pump designed as a roller pump, and an oxygen dispenser in the form of an oxygen concentrator connected on the drive side to the pump. The machine has a controller for the oxygen concentrator and the delivery rate of the pump and also connections for a decentralized energy supply and/or for an electrical energy store. Although this heart-lung machine is also intended for mobile use, it cannot normally be handled by one person on account of its size and weight. Owing to the confined space in an ambulance, it is virtually impossible to take this machine along on an emergency call-out in a ready-to-operate state. The haste required in emergency call-outs makes the long set-up and preparation times disadvantageous. In addition, after each time they are used, the aforementioned heart-lung machines have to be newly set up and cleaned in a complicated procedure before they are used again. As a result, immediate re-use is not possible.

WO 97/16213 (BAXTER) already discloses a mobile heart-lung machine in which the blood-circulating elements, including the blood pump, heat exchanger and oxygenator, are accommodated in an arrangement intended for once-only use and the pump motor is accommodated in a reusable drive unit. The arrangement intended for once-only use is connected to the drive unit by a force closure by means of a connecting part protruding high up from said drive unit. Further connections exist with the separate drive and automatic control units. The connections for the respective tubes to the arrangement are exposed and can be easily damaged or interrupted, in particular during mobile use.

It is therefore the object of the present invention to provide a mobile heart-lung machine of the type stated at the beginning which can be universally used and can be handled reliably and easily.

This object is achieved by the subject-matter of claim 1. The division into a module with elements which circulate the blood, receive the biochemical and physiological signals and implement the control signals (“disposable module”) and a module with drive and automatic control elements (“reusable module”) permits a rapid exchange of the module intended for once-only use, containing the blood-circulating elements, after use, so that the heart-lung machine can quickly be used again. On account of the modularity, it is additionally possible to produce the respective modules in an already serviceable and compact form. This ensures particularly high functional reliability in use, since no complicated operating and setting-up measures have to be carried out under the time pressure existing during use. Furthermore, the grouping together of the blood-circulating elements in a compact module allows the tubular blood-circulating system between the individual elements to be of a particularly short length, as a result of which the filling volume can be reduced, which is advantageous for the patient. Moreover, the routing and nature of the blood channels in this module can-be precisely defined and designed to provide favorable flow conditions, in order to reduce the blood trauma as much as possible. In this respect it is possible, for example, for commercially available components such as the oxygenator, centrifugal pump head, filter etc. to be connected by fixed blood channels. The individual components of the disposable module, designed as a cartridge, can be easily exchanged and replaced by others if need be.

The subclaims are directed at advantageous embodiments of the invention.

According to the invention, the two modules can be joined together in a simple way to form one unit, in that they can be fitted one on top of the other and can be connected mechanically and electrically to each other. The heart-lung machine is therefore easy to handle in mobile applications and can be carried by one person. This is also made possible by the blood pump being a centrifugal pump. Roller pumps are usually used as blood pumps. On account of their massive-pump element, they are heavy and require considerable energy during operation. Centrifugal pumps, on the other hand, are particularly economical in terms of energy. In addition, they are distinguished by a suction action on the basis of the negative pressure produced. As a result, the blood is sucked in to the extent required for supplying the patient, and according to the pumping rate, if an adequate supply by the passive return flow of blood alone cannot be ensured. This is important in particular when they are used in an emergency, if the cannulation of the patient has not been performed optimally or the tubes cannot be optimally arranged on account of outside circumstances.

According to the invention, the pump head through which the blood flows and the pump drive of the blood pump are respectively arranged in the corresponding modules. With the modules joined together, the pump head and the pump drive are connected to each other via a coupling. Since the elements coming into contact with blood are arranged in a single disposable module, designed as a sterile-packed unit, the production costs of the module intended for once-only use, and consequently the operating costs of the mobile heart-lung machine, can be kept low.

According to a preferred embodiment of the invention, the connections between the individual blood-circulating elements comprise flexible and/or rigid tubes to optimize and standardize the hemodynamics and to increase the internal rigidity. The blood-circulating components of the extracorporeal circuit and the tubes connecting them are fastened in the module housing by means of retaining or fastening devices. Such a system is more reliable than straightforward plug-in tube connections and contributes to the rigidity and better retention of the individual elements in a disposable module.

It has proven to be expedient for at least some of the blood-circulating elements to be formed in one piece with the module, for example as an injection molding. If the blood-circulating system or, for example, the housings of other blood-circulating elements in the module are an integral part of the disposable module, the latter can be further reduced in size and produced in an integrated form. It is also conceivable to make the housing of the module with the blood-circulating elements and the blood-circulating system in the form of a cartridge into which commercially available blood-circulating elements, such as the oxygenator, blood pump, pump head of a centrifugal pump, arterial filter or the like, can be fitted with the aid of fastening devices. Oxygenators, pumps, filters or similar blood-circulating elements with their own blood channels, gas-supply lines or the like, specially designed for use in the heart-lung machine according to the invention, can also be fitted into the cartridge, designed for example as an injection molding. This allows even greater integration and compactness of the disposable module to be achieved.

To provide an optical check on the blood flow at any time during the operation of the heart-lung machine, the disposable module has a transparent housing.

It has proven to be advantageous for the elements receiving the biochemical and physiological signals to be sensors which sense, inter alia, blood pH, blood pCO₂, blood pO₂, blood temperature, blood flow rate, FiO₂, gas temperature, gas flow rate, gas pO₂, gas pCO₂, water temperature, rotational speed of the pump or the current flow to the pump drive.

According to the invention, the sensors for sensing the parameters, for indicating purposes and for being used for acoustic, optical or acoustooptical warnings and for processing the values for control purposes are connected to the drive and automatic control system. They automatically control, inter alia, the pump drive of the blood pump and, in the event of deviations from the setpoint value of the blood flow, give acoustic, optical or acoustooptical warnings. In this way, any possible malfunctions and/or changes in the state of the patient are quickly and reliably indicated and appropriate action can be taken. For monitoring the blood flow, a flow sensor which quantitatively monitors the blood flow without coming into contact with the blood may also be arranged on the arterial tube. The pumping rate of the blood pump expediently lies between 2 l/min and 10 l/min and can be controlled infinitely variably or in discrete increments by means of the automatic control system.

According to a further embodiment of the invention, closable filling and venting openings which can be operated from the outside are provided on a reservoir arranged upstream of the blood pump for receiving venous blood, on the arterial filter and/or on the oxygenator, to allow the reservoir to be filled and vented completely and simply when the heart-lung machine is put into operation. They serve in particular for the feeding in of priming solution or medicaments, such as for example heparin for reducing coagulation. The reservoir may in this case be designed as a bag. Such a reservoir serves as a buffer, to compensate for a varying inflow and outflow of the blood. The use of a bag allows the reservoir to be adapted in a simple way to the amount of blood respectively to be received.

According to the invention, a temperature-control device may be provided on the oxygenator, for example in the form of a heat exchanger which can be operated with water and through which blood flows, in order to control the temperature of the blood as desired before it is fed back to the patient.

A pre-filled priming solution has the effect that the heart-lung machine is completely vented and kept on immediate standby before it is connected to the patient in circulatory operation. For this purpose, the blood-circulating system has a pre-bypass filter, which is connected as a bypass between the arterial tube and the venous tube and through which the priming solution circulates before it is operated for the patient. Furthermore, the blood circulation can be maintained through a bypass which can be operated from the outside in the possible event of clogging of the arterial filter.

In a development of the idea of the invention, the automatic control system has an input unit and an output unit for conducting a dialog with the user, in particular buttons/controls and/or a display, and also a program for initialization and functional control during the operation of the heart-lung machine, in order to ensure simple operator control.

The self-sufficient voltage supply provided may be designed according to the invention as a storage battery and have an indicator for its charging state. Imminent discharging of the storage battery can therefore be detected in good time, in order if need be to take measures for providing an external power supply. In the meantime, the pump could be operated by a mechanical hand crank.

The heart-lung machine advantageously also has a connection for an external voltage supply, to allow the storage battery to be charged for example in the ambulance or to permit operation with an external power supply when the storage battery has been depleted.

The invention is explained in more detail below on the basis of an exemplary embodiment represented in the drawing, in which:

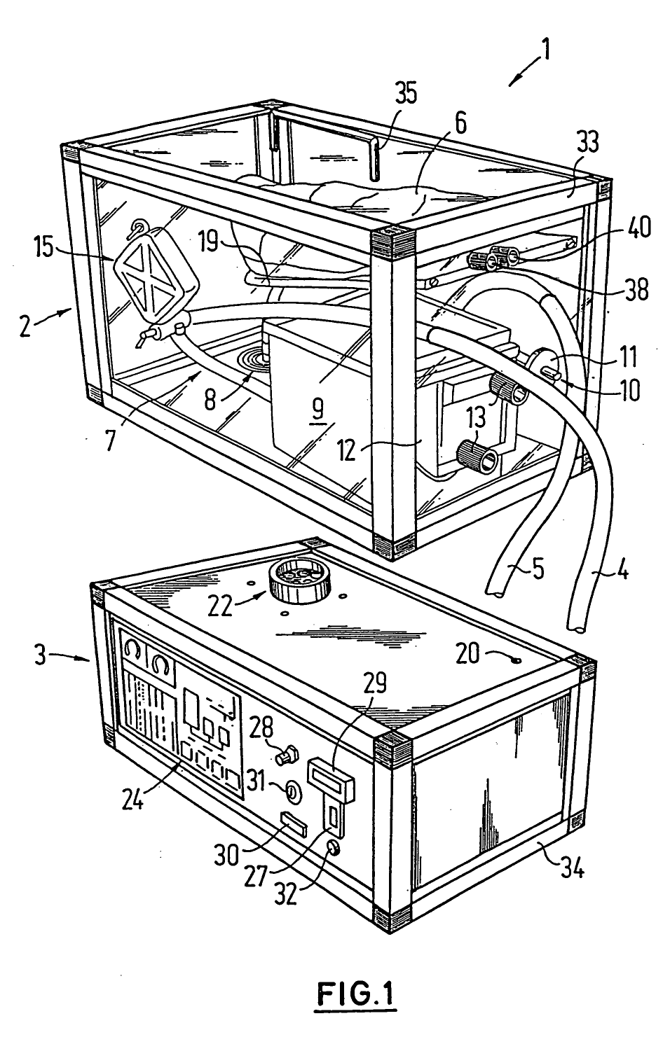

FIG. 1 shows a view obliquely from above of a mobile heart-lung machine according to the invention, with modules separated from each other,

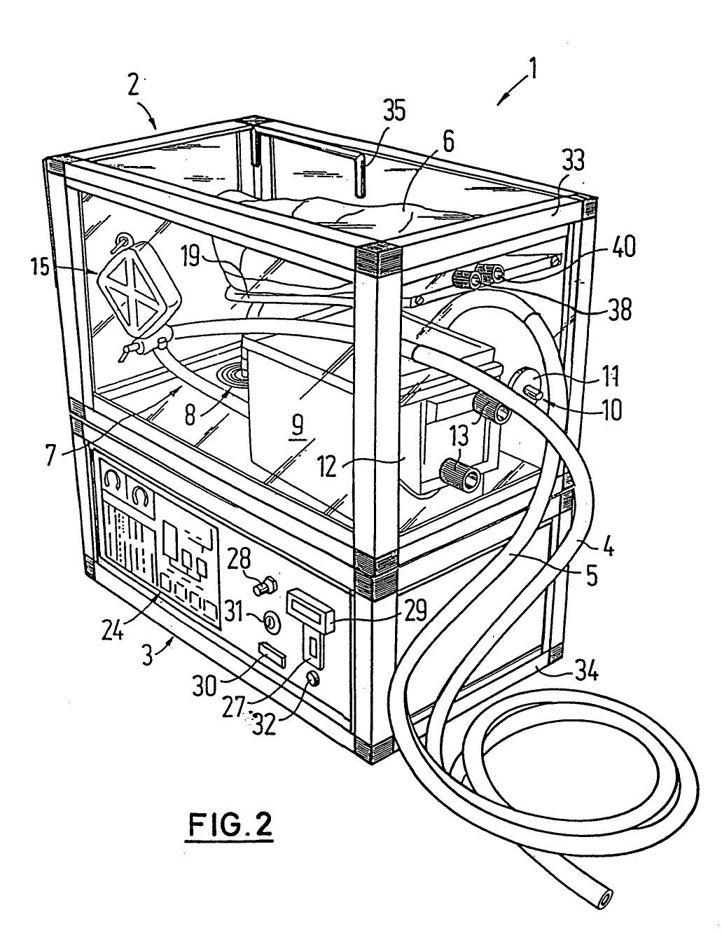

FIG. 2 shows a view of the heart-lung machine represented in FIG. 1, with the modules joined together,

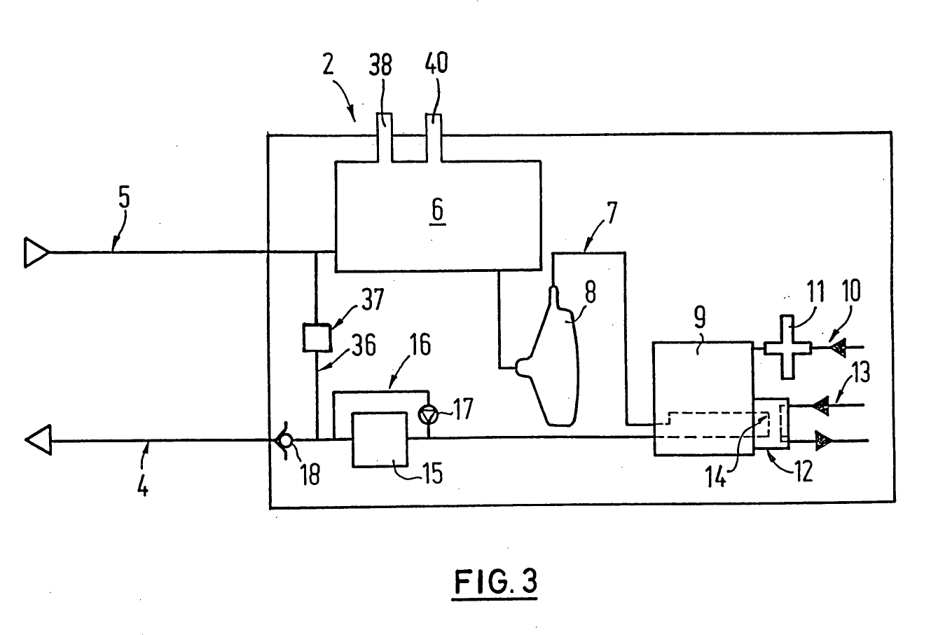

FIG. 3 shows a schematic representation of the blood-circulating elements of the heart-lung machine according to FIG. 1,

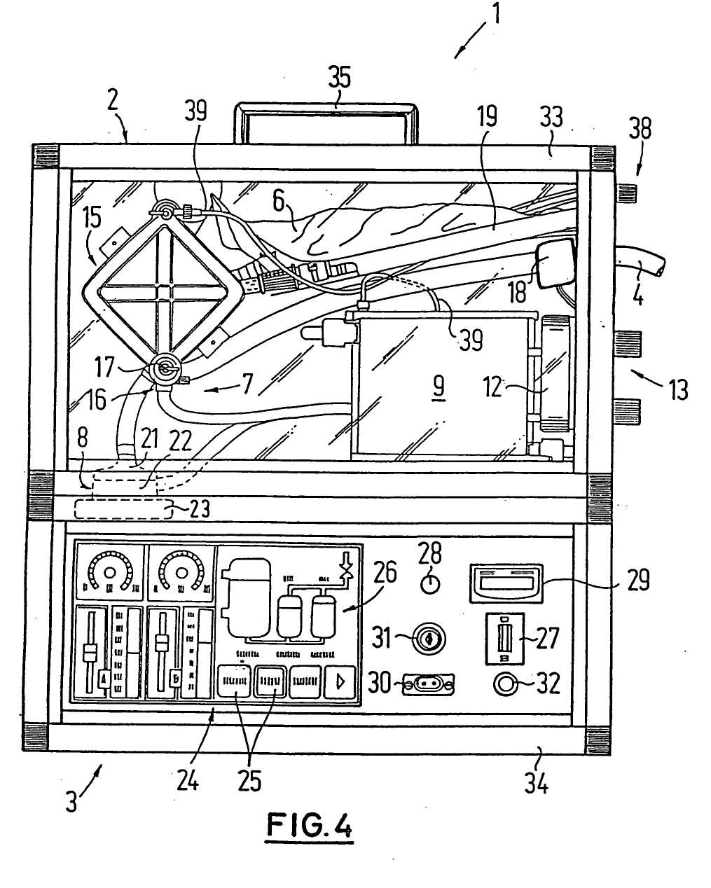

FIG. 4 shows a front view of the heart-lung machine according to FIG. 1,

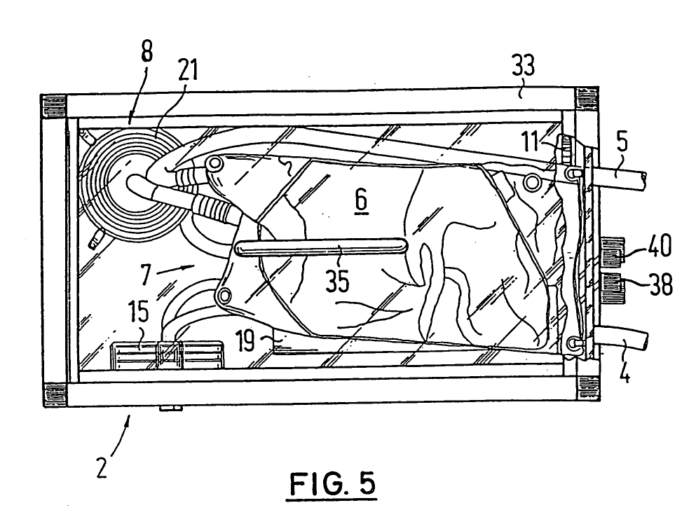

FIG. 5 shows a view from above of the heart-lung machine represented in FIG. 1 and

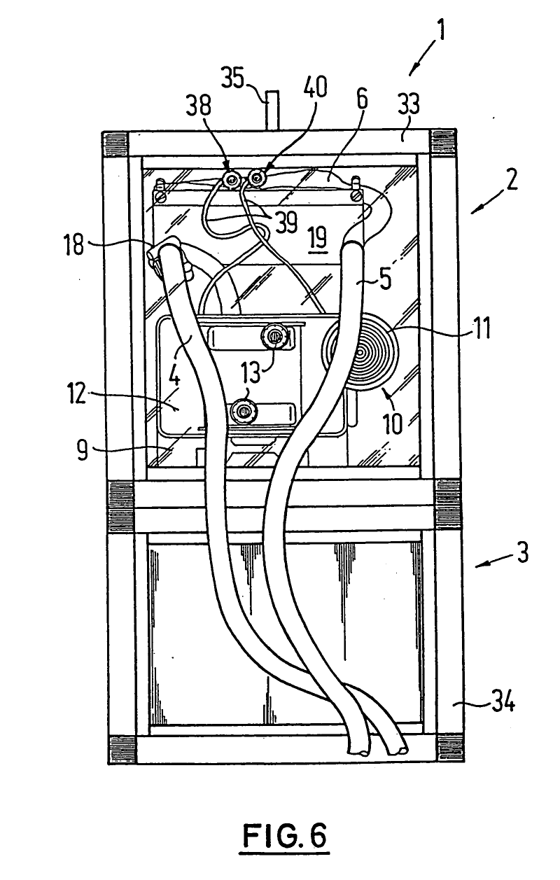

FIG. 6 shows a side view of the heart-lung machine according to FIG. 1.

The mobile heart-lung machine 1 shown in FIG. 1 comprises two separate modules 2 and 3. Accommodated in the first module 2 (“disposable module”), intended for once-only use, are the elements which circulate the blood, receive the biochemical and physiological signals and carry out the control commands, and accommodated in the second, reusable module 3 are the drive and control elements. The two modules 2 and 3 can be joined together to form one functional unit, be fitted one on top of the other and connected mechanically and at the same time electrically to each other by means of retaining elements (not represented) (cf. FIG. 2). In the joined-together state, the heart-lung machine 1 is ready for use after it has been filled with a priming solution, and can be connected by means of the arterial and venous tubes 4 and 5 to the blood circulation of the patient to be supplied.

The construction and mode of operation of the disposable module 2 is explained on the basis of the schematic representation in FIG. 3. Anoxemic blood of the patient is passed through a venous tube 5 to the module 2 of the heart-lung machine 1 containing the blood-circulating elements. The venous tube 5 may in this case be connected to a closed reservoir 6 for receiving venous blood. Through a blood-circulating system 7, the reservoir 6 is connected via a blood pump 8 to an oxygenator 9 for enriching the blood with oxygen and for eliminating carbon dioxide. A gas connection 10 opens into the oxygenator 9 through a filter 11 and serves for the connecting of an external gas supply. It is consequently possible to fall back on mobile oxygen and carbon dioxide cylinders or a hospital's oxygen supply system for the gas exchange in the oxygenator 9. Also arranged on the oxygenator 9 is a temperature-control device 12, which is flowed through by a temperature-control medium, through a circuit 13. For controlling the temperature of the blood, the blood-circulating system 7 has a portion 14 protruding into the temperature-control device 12. Arranged downstream of the oxygenator 9 in the blood-circulating system 7 is an arterial filter 15 for cleaning the blood. Provided around the arterial filter 15 is a bypass 16, which can be operated from outside by a valve 17, to allow the blood circulation to be maintained even in the possible event of clogging of the arterial filter 15. Connected to the outlet of the arterial filter 15 is the arterial tube 4 for returning the blood to the patient. For contactless monitoring of the blood flow, a flow sensor 18 which detects the blood flow and reports it to an automatic control system 24 is provided on the arterial tube 4.

FIG. 4 shows the front view of the heart-lung machine 1 with the disposable module 2, having the blood-circulating elements, and the reusable module 3 with the drive and control elements. Standing on the base of the disposable module 2 is the oxygenator 9 with the temperature-control device 12. The reservoir 6, designed as a bag, for receiving the venous blood is arranged above the oxygenator 9 on a sloping mount 19. Along with the oxygenator 9, the blood pump 8 is located on the base of the module 2. This blood pump is designed as a centrifugal pump and has a pump head 21, which is connected via a coupling 22 to a pump drive 23 arranged in the reusable module 3. The coupling 22 is a magnetic coupling. Arranged above the blood pump 8 is the arterial filter 15, which is connected to the arterial tube 4 and on which the bypass 16 with the valve 17 is located. Arranged just before the point where the arterial tube 4 leaves the module 2 is the contactless flow sensor 18. The blood-circulating elements are connected to one another via tubes of the blood-circulating system 7.

Arranged in the reusable module 3 is an automatic control system 24 with buttons 25 and a display 26.

The buttons 25 and the display 26 may also be designed in the form of a touch screen. In addition to the automatic control system 24, on the module 3 there is an indicator 27 for indicating the charging state of the storage battery, a controller 28 and a rotational speed indicator 29 for manually controlling the pumping rate of the centrifugal pump 8 and also a connection 30 for the connecting of an external voltage supply. Also provided are a central on/off switch 31 and a pilot lamp 32. In addition, the module 3 has a storage battery (not represented) for a self-sufficient voltage supply.

The disposable module 2 has a transparent module housing 33, while the reusable module 3 with the drive and control elements has an opaque module housing 34. Attached to the upper top surface of the transparent module housing 33 of the disposable module 2 is a carrying handle 35.

FIG. 5 shows the blood-circulating elements arranged in the disposable module 2 in plan view. The venous tube 5 is connected to the reservoir 6. By means of the blood-circulating system 7, the reservoir 6 is in connection through the blood pump 8 with the (hidden) oxygenator 9, the outlet of which opens via the arterial filter 15 into the arterial tube 4.

In FIG. 6, the heart-lung machine 1 is represented from the side, with the connections for the arterial and venous tubes 4 and 5. The outlets of the circuit 13 of the temperature-control device 12 and also the gas connection 10 with the filter 11 can also be seen.

Not represented is the connecting tube 36, schematically shown in FIG. 3, with the pre-bypass filter 37 between the venous tube 5 and the arterial tube 4. By opening closures which are not represented and can be operated from the outside, and by simultaneously clamping off the tubes 4 and 5, an internal circuit of the blood-circulating elements can be completed. In such circulating operation before connection of the heart-lung machine 1 to the patient, the blood-circulating elements can be completely vented by means of venting valves 38 and 40 on the reservoir 6. For this purpose, venting tubes 39 are also connected to the arterial filter 15 and the oxygenator 9.

The mode of operation of the mobile heart-lung machine 1 described above is as follows:

During the operation of the mobile heart-lung machine 1, anoxemic blood flows through the venous tube 5 into the reservoir 6. The feeding in of priming solution, medicaments or the like is possible through venting valves 38, 40 on the venous tube 5 and/or on the reservoir 6. From the reservoir 6, the blood is pumped by the centrifugal pump 8 into the oxygenator 9, in which it is enriched with oxygen. During the gas exchange in the oxygenator 9, the blood flows through the temperature-control device 12, in which the temperature of the blood can be controlled. For this purpose, a temperature-controlled medium, preferably water, can be fed into the temperature-control device 12 from the outside through the circuit 13. Oxygen or an oxygen mixture is introduced into the oxygenator 9 via a gas connection 10. Once it has been enriched with oxygen, the blood is passed for cleaning through the arterial filter 15, which is equipped with a bypass 16. Should the arterial filter 15 become clogged during operation, the bypass 16 can be manually opened in order to maintain the patient's blood circulation. The blood is subsequently fed back to the patient through the arterial tube 4, past-the flow sensor 18.

The flow sensor 18 measures the flow of the blood in the arterial tube 4 and is connected to the automatic control system 24 of the mobile heart-lung machine 1 via an electrical plug-in contact 20 (cf. FIG. 1). According to the flow measured, the automatic control system 24 controls the pump drive 23. When critical values are reached, the automatic control system 24 produces corresponding acoustic, optical or acoustooptical warnings.

Additionally provided in the disposable module 2 are further sensors (not represented) for receiving biochemical or physiological signals, which serve for checking the function of the heart-lung machine 1. The sensor signals are transmitted through contacts comparable to the plug-in contact 20 to the automatic control system 24, it being possible for all the contacts also to be grouped together in a multiple plug. This makes it easier for the two modules 2 and 3 to be joined together. Elements implementing the control signals, such as for example electrically activatable valves, may also be arranged in the disposable module 2 and be triggered by the automatic control system 24 via electrical contacts.

To disaccustom the patient from the heart-lung machine 1, the pumping rate of the blood pump 8 is continously reduced by means of the automatic control system 24 while checking and adapting the filling state of the heart. Only then can the heart-lung machine 1 be switched off, provided that the patient is again in a stable state. After switching off, the disposable module 2 with the blood-circulating elements can be separated in a simple way from the reusable module 3 with the drive and control elements by releasing the retaining elements and can be replaced by a new sterile disposable module 2. After closing the retaining elements and filling with priming solution, the heart-lung machine 1 is immediately ready for use again.

This provides a mobile heart-lung machine 1 which, on account of its modular construction, comprising a disposable module 2 and a reusable module 3, can be quickly put to use and, on account of the compactness of the two modules 2 and 3, can be handled by one person and, consequently, is particularly suitable for emergency call-outs.

By adding a suction-device/cardioplegia management system for venting, suction and stabilizing of the heart, the mobile heart-lung machine 1 is also suitable for stationary use in hospitals, for example for heart surgery.

List of Designations

1 Mobile heart-lung machine
2 Module with blood-circulating elements
3 Module with drive and control elements
4 Arterial tube
5 Venous tube
6 Reservoir
7 Blood-circulating system
8 Blood pump
9 oxygenator
10 Gas connection
11 Filter
12 Temperature-control device
13 Circuit
14 Portion
15 Arterial filter
16 Bypass
17 Valve
18 Flow sensor
19 Mount
20 Plug-in contact
21 Pump head
22 Coupling
23 Pump drive
24 Automatic control system
25 Buttons
26 Display
27 Indicator
28 Controller
29 Rotational speed indicator
30 Connection
31 On/off switch
32 Pilot lamp
33 Transparent module housing
34 Module housing
35 Carrying handle
36 Connecting tube
37 Pre-bypass filter
38 Venting opening or valve
39 Venting tubes
40 Venting opening or valve

1. A mobile heart-lung machine for maintaining circulation by taking over or supporting the cardiopulmonary function, with at least one venous tube (5), a blood pump (8), an oxygenator (9), an arterial filter (15), an arterial tube (4), and also a tubular blood-circulating system (7), an automatic control system (24) and a self-sufficient voltage supply, characterized in that the elements (4, 5, 6, 7, 8, 9, 15, 18, 21) which circulate the blood, receive the biochemical and physiological signals and carry out the control commands on the one hand and the drive and automatic control elements (23, 24, 27, 28, 29, 30, 31, 32) on the other hand are arranged in two separate modules (2, 3) and can be joined together to form one functional unit.

2-14. (Cancel)
